(19) United States
(12) Patent Application Publication
(10) Pub. No.: US 2010/0048710 A1
(43) Pub. Date: Feb. 25, 2010

(54) Methods and Compositions for Modulating Muscle Fat in Livestock

(76) Inventors: Daniel Buskirk, Mason, MI (US); Matthew E. Doumit, Mason, MI (US); Guillermo Ortiz-Colon, Mayaguez, PR (US)

Correspondence Address: MEDLEN & CARROLL, LLP, 101 HOWARD STREET, SUITE 350, SAN FRANCISCO, CA, 94105, US

(21) Appl. No.: 12/226,846

(22) PCT Filed: May 1, 2007
(86) PCT No.: PCT/US2007/010526

§ 371 Date: Jun. 2, 2009

Related U.S. Application Data
(60) Provisional application No. 60/796,501, filed on May 1, 2006.

(51) Int. Cl. A61K 31/192; A61P 29/00

(52) U.S. Cl. 514/570

(57) ABSTRACT

The present invention relates to compositions and methods for modulating the degree of adipose tissue deposited intramuscularly in livestock animals. In particular, the present invention relates to compositions and methods for the use of non-steroidal antiinflammatory drugs to preferentially increase the amount of intramuscular adipose tissue in cattle.

This application claims priority to U.S. Provisional Application No. 60/796,501 filed May 1, 2006, which is incorporated herein in its entirety.

### FIELD OF THE INVENTION

The present invention relates to compositions and methods for modulating the degree of adipose tissue deposited intramuscularly in livestock animals. In particular, the present invention relates to compositions and methods for the use of non-steroidal anti-inflammatory drugs to preferentially increase the amount of intramuscular adipose tissue in cattle.

### BACKGROUND OF THE INVENTION

Marbling is the common name used to describe the adipose tissue (i.e. fat) that is embedded in the connective tissue of livestock animals (e.g., beef cattle). Independent of its physiological function, the accretion of intramuscular fat is desirable in domestic cattle because it is positively associated with the palatability of beef, and is the main determinant of USDA Quality Grade within a carcass maturity classification (Kerth et al., 1999, J. Anim. Sci. 77:116-119; Wheeler et al., 1999, J. Anim Sci. 77:882-888). Although beef cattle are currently fed high energy diets for extended periods of time in an attempt to assure a desirable degree of marbing, the National Beef Quality Audit-2000 found that insufficient development of marbling and the excess deposition of subcutaneous fat (i.e. fat found under the skin) were among the top challenges of the industry. Further, excessive development of adipose tissue, other than intramuscular adipose tissue, is costly; it is not unusual for 30% of the weight of a beef carcass to be waste adipose tissue (Smith, 1995, The Biology of Fat in Meat Animals: Current Advances, Amer. Soc. Of Anim. Sci, Champain, Ill., pp. 166-168).

As such, there is a great need in the livestock industry for the development of materials and methods that would preferentially increase the deposition of adipose tissue intramuscularly in livestock.

### SUMMARY OF THE INVENTION

Adipogenesis is the formation of adipose cells from mesodermal stem cells. Unipotent mesodermal cells committed to the adipogenic lineage are referred to as adipoblasts. Adipoblast growth arrest at the G1/S mitotic boundary is critical for the cells to enter the preadipocyte state (Dani, et al., 1989, J. Biol. Chem. 264:10119-10125; Smas and Sul, Biochem J. 309:697-710; Gregoire et al., 1998, Physiol. Rev. 78:783-809). Preadipocytes already expressing early differentiation markers, but without considerable triacylglyceride stores, then undergo at least one round of DNA replication (Ailhaud, 2001, Adipose Tissues, Eurekah Publ, pp. 27-55). This process is known as clonal amplification of committed cells. After clonal expansion, preadipocytes enter into an irreversible growth arrest stage at the interval between mitotic stage G1 and differentiation (GD) (Ntambi and Kim, 2000, J. Nutr. 130:3122S-3126S).

Peroxisome proliferator-activated receptor γ2 (PPAR γ2) is considered the master regulator of adipogenic gene expression (Schoonjans et al., 1996, Biochim. Biophys. Acta 1302:93-109; Tamori et al., 2002, Diabetes 51:2045-2055; Knouff and Auwerx, 2004, Endocr. Rev. 25:899-918; Rosen, 2005, Prost., Leuko., Essent Fatty Acids 73:31-34). Peroxisome proliferator-activated receptor γ2 (PPARγ2) forms heterodimers with the retinoic X receptor-α (RXRα) that, in the basal state, is bound to co-repressor proteins (Knouff and Auwerx, 2004; Miard and Fajas, 2005, Int. J. Obes. 29:S10-S12). The transcriptional activity of PPARγ2 is increased by ligands (Brun et al., 1996, Curr. Opin. Cell. Biol. 8:826-832). Upon binding, these ligands cause PPARγ2 to change its conformation, which results in the release of co-repressors and recruitment of co-activators (Knouff and Auwerx, 2004). Because the type of ligand determines which co-activators are recruited by the PPARγ2-RXRα heterodimer, and the co-activators determine the target genes of PPARγ2 (Yu et al., 1995, J. Biol. Chem. 270:23975-23983; Debril et al., J. Mol. Med. 79:30-47; Houseknecht et al., 2002, Domest. Anim. Endo. 22:1-23; Bishop-Bailey and Wray, 2003, Prost. and other Lipid Mediat. 71:1-22; Miard and Fajas, 2005), the type of ligand available is important in the regulation of adipogenesis.

Ibuprofen (IBU), a non-steroidal anti-inflammatory drug (NSAID), is a well established inhibitor of cycloxygenase (COX) activity (Rome and Lands, 1975, Proc. Natl. Acad. Sci. 72:4863-4865; Mitchell et al., 1993, Proc. Natl. Acad. Sci. 90:11693-11697) and has been characterized as a PPARγ2 ligand (Lehmann et al., 1997, J. Biol. Chem. 272:3406-3410). Ibuprofen induces preadipocyte differentiation in 1246 preadipocytes (Ye and Serrero, 1998, Biochem. J. 330:803-809) and C3H10T1/2 clone 8 fibroblasts (Lehmann et al., 1997). However, at concentrations compatible with COX inhibition (Rome and Lands, 1975), preadipocyte differentiation was not enhanced in C3H10T1/2 clone 8 fibroblasts. Another COX inhibitor, indomethacin, has also been shown to have adipogenic effects that are independent of COX inhibition (Knight et al., 1987, Mol. Endocrin. 1:36-43).

Ibuprofen has been reported to induce the expression of adipophilin (Ye and Serrero, 1998). Because adipophilin enhances the uptake of long chain fatty acids and is involved in the intracellular storage of neutral lipids, its inhancement by IBU could be important in preadipocyte differentiation (Imamura et al., 2002, Am. J. Physiol. Endo. Metab. 283:E775-E783). The adipophilin gene has been reported to have PPARγ2 elements (Targett-Adams et al., 2005, Biochim. Biophys. Acta 1728:95-104) and IBU enhances the transcription of adipophilin (Ye and Serrero, 1998). This strongly suggests that IBU enhances preadipocyte differentiation by functioning as a PPARγ2 ligand (Lehmann et al., 1997).

Ibuprofen may induce preadipocyte differentiation through PPARγ2 independent mechanisms. Ibuprofen has been shown to inhibit nuclear factor kappa β (NF-κβ) stimulation of gene transcription (tegeder et al., 2001, FASEB J. 15:2057-2072). One of the genes stimulated by NF-κβ is tumor necrosis factor α (TNFα) (Clark and Lasa, 2003, Curr. Opin. Pharma. 3:404-411). Tumor necrosis factor α rapidly decreases PPAR γ2 mRNA and protein, and PPAR γ2 DNA binding activity (Gregoire et al. 1998), and also induces interleukin-6 expression in preadipocytes and adipocytes, which decreases lipoprotein lipase expression (Fried et al., 1998, J. Clin. Endo. Metab. 83:847-850). In addition, TNFα is linked to insulin resistance, inhibition of glucose intake and inhibition of preadipocyte differentiation (Fruhbeck et al., 2001, Am. J. Physiol. Endo. Metab. 280:E827-E847).

The present invention is not limited to a particular mechanism. Indeed, an understanding of the mechanism is not necessary to practice the present invention. Nonetheless, we contemplate that bovine intramuscular (IM) and subcutaneous (SC) preadipocytes differ in their ability to accumulate lipid. Ibuprofen increases adipogenesis in IM preadipocytes, under conditions that it functions as an activator of PPARγ2. The differences in adipogenic capacity among IM and SC preadipocytes are related to differences in the endogenous activation of PPARγ2. It is contemplated that the preferential enhancement of adipogensis in IM preadipocytes by IBU will increase IM lipid accretion in livestock (e.g., cattle) with little or no increase in SC fat accretion.

Therefore, in one embodiment, the present invention is a method for modulating the intramuscular fat content of a livestock animal comprising providing a solution containing a non-steroidal anti-inflammatory drug or analog thereof, a livestock animal, and delivering said solution to said livestock animals thereby causing an increase in intramuscular fat content. In some embodiments, the non-steroidal anti-inflammatory drug or analog thereof is ibuprofen. In some embodiments, said livestock animals are cattle.

### DESCRIPTION OF THE FIGURES

FIG. 1 shows the effect of dexamethasone (DEX), ibuprofen (IBU), and troglitazone (TGZ) on the activity of glycerol-3-phosphate dehydrogenase (GPDH) in bovine clonal preadipocytes isolated from suncutaneous adipose tissue

FIG. 2 shows the effect of dexamethasone (DEX) and ibuprofen (IBU) on the activity of glycerol-3-phosphate dehydrogenase (GPDH) in bovine clonal and heterogeneous preadipocyte cultures isolated from intramuscular (IM) and subcutaneous (SC) adipose tissue.

FIG. 3 shows the effect of dexamethasone (DEX) and ibuprofen (IBU) on the activity of glycerol-3-phosphate dehydrogenase (GPDH) in bovine heterogeneous preadipocyte cultures isolated from intramuscular (IM) and subcutaneous (SC) adipose tissue.

### DEFINITIONS

The term “livestock” as used herein refers to any animal where a modulation in intramuscular adipose tissue is desirable. For example, livestock of the present invention are preferentially cattle, such as beef, reindeer, elk, buffalo, etc.

The term “non-steroidal anti-inflammatory drug” (NSAID) as used herein refers to any drug that inhibits the cyclooxygenase (COX) enzyme. For example, ibuprofen, piroxicam, and aspirin are NSAIDs. Analogs of NSAIDs also fall within the scope of this definition.

The term “intramuscular” as used herein refers to within, or in close approximation with, a muscle of a livestock animal.

The term “subcutaneous” as used herein refers to under the skin or dermal layer of an animal, not in association with a muscle of a livestock.

The term “test animal” as used herein refers broadly to any animal. Examples of test animals include, but are not limited to mice, rats, guinea pigs, cats, dogs, ungulates (e.g., sheep, goats, cows, etc.).

### DESCRIPTION OF THE INVENTION

The present invention relates to compositions and methods for modulating the degree of adipose tissue deposited intramuscularly in livestock animals. In particular, the present invention relates to compositions and methods for the use of non-steroidal anti-inflammatory drugs to preferentially increase the amount of intramuscular adipose tissue in cattle.

The present invention is not limited to a particular mechanism. Indeed, an understanding of the mechanism is not necessary to practice the present invention. Nonetheless, we contemplate that bovine IM and SC preadipocytes differ in adipogenic capacity while equally expressing PPARγ2, a key regulator of adipogenesis. Because PPARγ2 function depends on its activation by ligands, it is contemplated that adipogenic differences between IM and SC preadipocytes are related to differences in the synthesis of biologically relevant PPARγ2 ligands, other than prostacyclin. Supplementation with an exogenous PPARγ2 ligand, troglitazone (TGZ), equally stimulates differentiation of bovine IM and SC preadipocytes (Grant, 2005). Conversely, supplementation with the PPARγ2 ligand indomethacin induces angiogenesis in bovine omental (OM) preadipocytes when compared to SC preadipocytes, which results in the elimination of their adipogenic differences (Wu et al., 2000). The present invention is not limited to a particular mechanism. Indeed, an understanding of the mechanism is not necessary to practice the present invention. Nonetheless, we contemplate that specific PPARγ2 ligands may selectively enhance adipogenesis of bovine IM preadipocytes, and thereby selectively stimulate IM fat development (e.g., marbling).

Ibuprofen is a non-steroidal anti-inflammatory drug known to inhibit cyclooxygenase, and is a known PPARγ2 ligand. Ibuprofen is used to induce adipogenesis in murine adipocytes (Ye and Serrero, 1998). The present invention is not limited to a particular mechanism. Indeed, an understanding of the mechanism is not necessary to practice the present invention. Nonetheless, we contemplate that bovine IM preadipocytes have a limited adipogenic capacity as a result of limited syntheseis of biologically relevant

PPARγ2 ligands, and that IBU will enhance adipogeneis of bovine IM preadipocytes.

Cyclooxygenase is the enzyme that catalyzes the rate limiting rection during the biosynthesis of prostanoids. Prostanoids have been associated positively and negatively with adipogenesis. The present invention is not limited to a particular mechanism. Indeed, an understanding of the mechanism is not necessary to practice the present invention. Nonetheless, we contemplate that IBU inhances preadipocyte adipogenesis by indirectly inhibiting the expression of an anti-adipogenic protein TNFα. TNFα inhibits preadipocyte adipogenesis by decreasing the expression of PPARγ2 and lipoprotein lipase, and by inducing insulin resistance. Ibuprofen inhibits NF-κβ stimulation of gene transcription, and TNFα is one of the genes stimulated by NF-κβ. Aspirin has also been reported to inhibit NF-κβ, but does not stimulate preadipocyte differentiation or activate PPARγ2. Aspirin (500 μM) did not affect GPDH activity in either preadipocyte population (Example 5 results). The present invention is not limited to a particular mechanism. Indeed, an understanding of the mechanism is not necessary to practice the present invention. Nonetheless, the data indicate that IBU induces bovine IM preadipoctye adipogenesis independently of COX inhibition and NF-κβ signaling by acting as a PPARγ2 ligand.

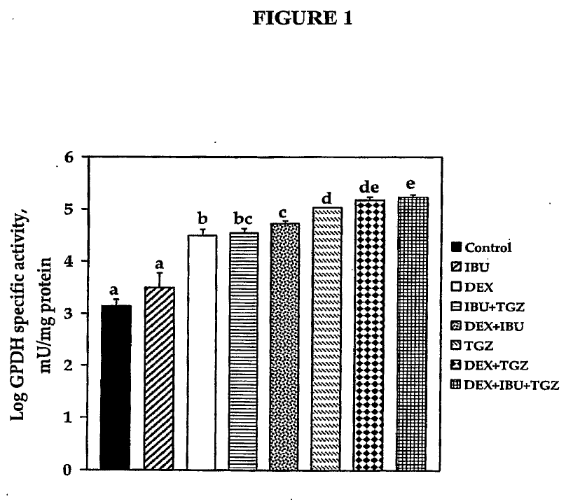

An additional PPARγ2 ligand, TGZ, equally increases adipogenesis in bovine IM and SC preadipocytes. Initial comparisons between the effects of IBU and TGZ on adipogenesis of clonal SC preadipocytes were performed. In contrast to TGZ, IBU incubation for 48 hours did not enhance the GPDH activity of bovine SC clonal preadipocytes without concomitant exposure to DEX. As well, IBU suppressed TGZ stimulation of GPDH activity when DEX was not included in the experiment (FIG. 1). Indeed, an understanding of the mechanism is not necessary to practice the present invention. Nonetheless, the data indicate that IBU stimulates bovine preadipocyte differentiation through a different mechanism than TGZ.

TGZ is a member of the thiazolidinedione family of antidiabetic drugs characterized as PPARγ2 ligands, and TGZ also has a non-genomic, PPARγ2 independent mechanisms of action. TGZ activates phosphatidylinositol 3-kinase (PI3K), which subsequently activates various mitogen activated protein kinases (MAPK). PI3K activates extracellular signal regulated kinase (ERK) that in turn activates MAPK phosphatase-1 (MKP-1). As an inhibitor of MAPK, MKP-1 triggers cells cycle withdrawal, an essential step for the progression of preadipocyte differentiation. Inhibiton of PI3K prevents differentiation of 1246 and 3T3-L1 preadipocytes. The present invention is not limited to a particular mechanism. Indeed, an understanding of the mechanism is not necessary to practice the present invention. Nonetheless, it is contemplated that the rapid, PPARγ2 independent activation of PI3K by TGZ is a crucial difference between IBU and TGZ mechanisms of action.

The IBU inhibition of TGZ enhancement of bovine preadipocyte differentiation supports the observation that IBU acts as a PPARγ2 ligand. Indomethacin, a PPARγ2 activator, stimulates preadipocyte differentiation and acts as a ligand of higher activity. The present invention is not limited to a particular mechanism. Indeed, an understanding of the mechanism is not necessary to practice the present invention. Nonetheless, it is contemplated that IBU, which has been described as a low activity ligand, can act as a PPARγ2 activator thereby enhancing preadipocyte differentiation.

Various PPARγ2 ligands have the ability to induce PPARγ2 to recruit different co-activators, and these co-activators induce PPARγ2 target genes. The present invention is not limited to a particular mechanism. Indeed, an understanding of the mechanism is not necessary to practice the present invention. Nonetheless, it is contemplated that IBU preferentially enhances adipogenesis of bovine IM preadipocytes by recruiting PPARγ2 co-activators that are differentially expressed in IM preadipoctyes. Therefore, IBU increases adipogenesis in bovine IM adipocytes compared to SC adipocytes, and that adipogenic differences between IM and SC preadipocytes are related to differences in the endogenous activation of PPARγ2.

In one embodiment, the present invention relates to methods of introducing a COX inhibitor to livestock to increase intramuscular adipose tissue. In some embodiments, the COX inhibitor is from the family of non-steroidal anti-inflammatory drugs (e.g., NSAIDs, for example, ibuprofen, aspirin, naproxen sodium, carprofen, peroxicam, etc.). In some embodiments, the NSAID delivered is 2-(p-isobutylphenyl)propionic acid (e.g., ibuprofen). In some embodiments, the COX inhibitor is an analog of a COX inhibitor. For example, U.S. Pat. No. 3,228,831 lists analogs of 2-(p-isobutylphenyl)propionic acid, and is incorporated herein in its entirety. In some embodiments, the livestock is any animal where increased intramuscular adipose deposition is desirable for nutritional and/or economic reasons. In some embodiments, the COX inhibitor is delivered to cattle (e.g., beef, buffalo, elk, reindeer, water buffalo, etc.). In some embodiments, the delivery of the COX inhibitor in to beef cattle. In some embodiments, the beef cattle are male (e.g., steer).

In one embodiment, the delivery of the COX inhibitor is in conjunction with food stuffs. In some embodiments, the COX inhibitor is incorporated into feed for animal consumption. In some embodiments, the COX inhibitor is added to the feed in a liquid or solid form. In some embodiments, the COX inhibitor is given in a concentration sufficient to promote adipose tissue deposition intramuscularly. In some embodiments, the concentration of the COX inhibitor given to the animal is between 1 and 50 mg/kg body weight. In some embodiments, the delivery of the COX inhibitor is by injection either intravenous, intramuscular, or parenterally. In some embodiments, the COX inhibitor is given by injection in a concentration sufficient to promote adipose tissue deposition intramuscularly.

In one embodiment, the COX inhibitor or analog thereof is delivered in solution with buffers, solutions, chemicals, and adjuvants that increase the stability of the drug. In some embodiments, the COX inhibitor or analog thereof is incorporated into vitamins or other supplements that are fed to livestock. In some embodiments, the COX inhibitor or analog thereof is administered orally in aqueous form (e.g., dissolved or in liquid form mixed with water, etc.).

In one embodiment, the present invention relates to methods of determining COX inhibitors that modulate intramuscular deposition of adipose tissue. In some embodiments, the methods are performed in vitro and include a test compound and tissue culture cells. In some embodiments, the test compound is suspected of being a COX inhibitor or an analog thereof. In some embodiments, the method of detecting adipose tissue deposition includes fluorescence, luminescence, radiometry, or colorimetry. In some embodiments, the method for determining COX inhibitors that modulate intramuscular deposition of adipose tissue are performed in vivo in a test animal. In some embodiments, the in vivo method of detection includes fluorescence, luminescence, radiometry, or colorimetry.

Those skilled in the art would recognize additional avenues of administration of a COX inhibitor or analog thereof to livestock. Indeed, the present invention is not limited to the mode of administration of a COX inhibitor or analog thereof.

The following examples serve to illustrate certain embodiments and aspects of the present invention and are not to be construed as liming the scope thereof.

### Example 1

### General Cell Culture Techniques

Unless otherwise stated, all reagents were tissue culture grade and were purchased from Sigma (St. Louis, Mo.).

Preadipocytes from IM and SC adipose tissue were isolated using a modification of Forest et al., 1987, Exp. Cell Res. 168:218-232. Cells were seeded at a density of 4,160 cells/cm² in 35 mm cell culture wells in DMEM with 5.5 mM glucose, supplemented with 100 units/ml penicillin G, 0.1 mg/ml streptomycin sulfate, 0.25 μg/ml amphotericin B, 0.05 mg/ml gentamicin, 33 μM biotin, 17 μM pantothenate, 200 μM ascorbate, 1 mM octanoate, and 10% fetal bovine serum. The cells were grown in a 37° C./5% CO₂ incubator. Growth media was replaced every two days until the cells reached confluency (approximately 4 days), at which point the cells were washed twice with phosphate buffered saline (PBS) and differentiation treatments were applied. The differentiation medium consisted of DMEM with 5.5 mM glucose, 100 units/ml penicillin G, 0.1 mg/ml streptomycin sulfate, 0.25 μg/ml amphotericin B, 0.05 mg/ml gentamicin, 33 μM biotin, 17 μM pantothenate, 200 μM ascorbate, 280 nM bovine insulin, and 5 μl/ml bovine serum lipids (Ex-Cyte, Serologicals Corp., Norcross, Ga.). After 48 hours of differentiation media exposure, the cells were washed twice with PBS and differentiation media was replaced every two days for 10 days.

Glycerol-3-phosphate dehydrogenase (GPDH) activity was quantified biochemically by measuring GPDH enzyme activity using a modification of a previously published method (Adams et al., J. Clin. Invest. 100:3149-3153).

### Example 2

### Treatment of Bovine Preadipocytes with Ibuprofen (IBU), Dexamethasone (DEX), and Troglitazone (TGZ)

Clonal bovine SC cells were grown to confluency and exposed to unsupplemented differentiation media (control) or differentiation media supplemented with 25 nM DEX, 100 μM IBU, or 40 μM TGZ, or combinations thereof for 48 hours. Troglitazone was solubilized in ethanol (2.5 mg/ml), therefore treatments not containing TGZ were supplemented with an equivalent concentration of ethanol. After 48 hours, the media was replaced with fresh differentiation media, and fresh media was replaced every two days thereafter for 10 days. Each treatment was performed in duplicate. Adipogenesis was quantitated by measuring GPDH activity.

### Example 3

### Comparison of Effects of Ibuprofen on Intramuscular Versus Subcutaneous Adipogenesis

Cells were grown to confluence and exposed to unsupplemented differentiation media (control) or differentiation media supplemented with 25 nM DEX and IBU at 0, 250, 500, 1000, or 2000 μM concentrations for 48 hours. After treatment for 48 hours, media was changed as previously described until day 10. Cells from two beef steers were used, and duplicates of each steer treatment were done. Adipogenesis was quantitated by measuring GPDH activity.

### Example 4

### Comparison of the Effects of Increased Ibuprofen Exposure on Bovine IM Versus SC Adipogenesis

Heterogeneous IM and SC preadipocytes were grown to confluency and exposed to unsupplemented differentiation media or differentiation media supplemented as described in Example 2, except that the dose of IBU did not include 2000 μM concentration. Media was changed every two days as previously described until day 12. Adipogenesis was quantitated by measuring GPDH activity.

### Example 5

### Comparison of the Effects of Ibuprofen, Aspirin, and Indomethacin on Bovine IM and SC Adipogenesis

Heterogeneous IM and SC preadipocytes were gown to confluency and exposed to unsupplemented differentiation media or differentiation media supplemented with IBU concentrations as described in Example 3, or aspirin (ASP) or indomethacin (IND) concentrations of 0 or 500 μM. Media was changed every two days as previously described until day 12. Adipogenesis was quantitated by measuring GPDH activity.

### Example 6

### Statistical Analysis

Data were analyzed using the mixed model procedure of SAS (SAS, Cary, N.C.). In all experiments, pooled cells from two wells of a 6 well (35 mm) plate were considered the experimental unit. When main effects were significant (P<0.05), differences between means were evaluated utilizing Tukey's multiple comparison test. In Example 2, data means were calculated using the fixed effects of DEX, IBU, and TGZ. In Examples 3 and 4, data means were calculated using the fixed effects of DEX and IBU and their interactions with steer and steer x replication included as random variables. In Example 5, data means were calculated using the fixed effects of DEX, IBU, ASP, IND and their interactions with steer and steer x replication included as random variables. To satisfy the conditions of normality and homogeneity of variance, GPDH data were logₑ transformed in Example 2, and GPDH data were square root transformed in Example 3.

### Results

As can be seen in FIG. 1, addition of 100 μM IBU to differentiation media for 48 hours did not enhance clonal SC preadipocyte GPDH activity over control levels (P=0.99). Conversly, TGZ enhanced GPDH activity over control (P<0.001). Concomitant exposure of IBU and TGZ diminished TGZ stimulation of differention (P<0.001). DEX (25 mM) increased GPDH activity significantly, and this effect was enhanced by the concomitant exposure to IBU. DEX and TGZ did not have additive effects. The combination of DEX, IBU and TGZ resulted in higher GPDH activity than obtained with TGZ alone, but similar GPDH activity to cells treated with DEX and TGZ.

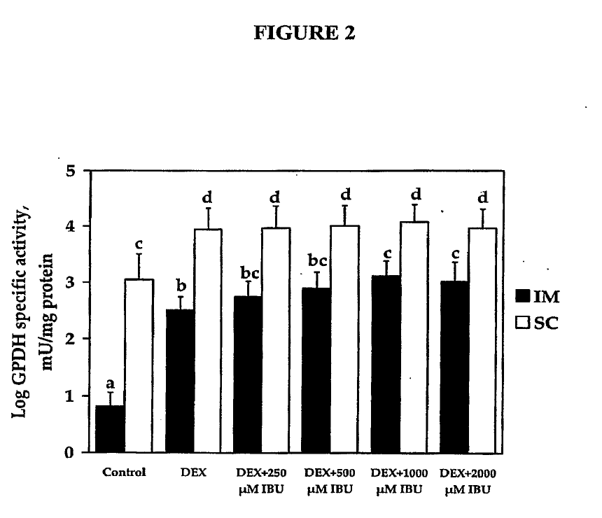

Subcutaneous preadipocytes were more adipogenic than IM preadipocytes (P<0.01) and DEX enhanced GPDH activity in both preadipocyte populations (P<0.001). Exposure to IBU for 48 hours did not enhance DEX stimulation of GPDH activity in SC preadipocytes. Conversely, exposure to 1000 or 2000 μM IBU enhanced DEX stimulation of GPDH activity in IM preadipocytes (FIG. 2).

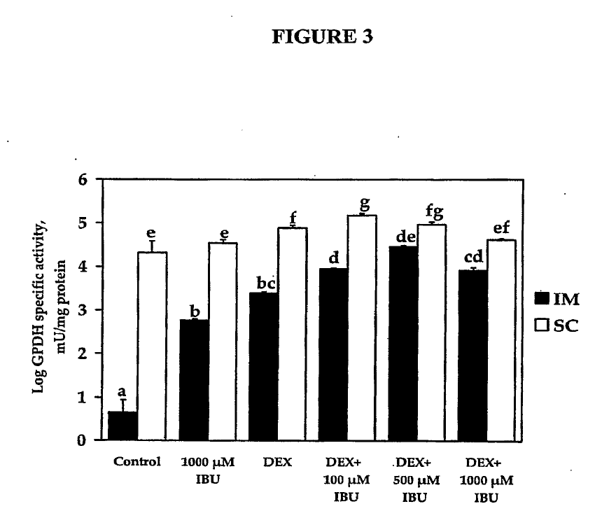

As seen in FIG. 3, 12 day exposure (long term) of bovine preadipocytes to IBU resulted in a treatment interaction. Exposure to 100 and 500 μM IBU enhanced DEX induction of differentiation in IM preadipocytes, whereas only 100 μM IBU enhanced DEX induction of differentiation in SC cells. Increasing the concentration of IBU to 1000 μM reduced DEX stimulation of GPDH in SC, but not IM preadipocytes.

For Example 5, in the absence of DEX exposure of bovine preadipocytes to IBU for 12 days resulted in a treatment interaction (P<0.001). Exposure to 10, 100, and 500 μM IBU enhanced GPDH activity in SC preadipocytes (P<0.001), whereas 500 μM IBU enhanced GPDH in IM preadipocytes. At 1000 μM IBU the activity of GPDH was higher than control only in IM cells (P=0.003). Under control conditions, SC preadipocytes had higher GPDH activity than IM preadipocytes (P<0.001). The maximum induction of GPDH activity by IBU was much greater in IM than SC cells (12 fold vs. 1.7 fold over control, respectively). Aspirin (500 μM) did not enhance GPDH activity either alone or in combination with DEX in either cell population. Indomethacin was toxic to cells.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in biochemistry, molecular biology, plant biology, and chemistry or related fields are intended to be within the scope of the following claims.

We claim:

1. A method for modulating the intramuscular fat content of a livestock animal comprising:
a) providing a COX inhibitor or analog thereof,
b) providing a livestock animal, and
c) delivering said COX inhibitor or analog thereof to said livestock animal thereby causing an increase in intramuscular fat content.

2. The method of claim 1, wherein said COX inhibitor or analog thereof is ibuprofen.

3. The method of claim 1, wherein said livestock animal is cattle.
